(19) United States
(12) Patent Application Publication
(10) Pub. No.: US 2016/0166785 A1
(43) Pub. Date: Jun. 16, 2016

(54) MONITORING OF NEBULIZER USAGE

(71) Applicant: KONINKLIJKE PHILIPS N.V., AE Eindhoven (NL)

(72) Inventors: MARK STEVEN MORRISON, BASKING RIDGE, NJ (US); DIRK ERNEST VON HOLLEN, CLARK, NJ (US)

(21) Appl. No.: 14/903,704

(22) PCT Filed: Jun. 27, 2014
(86) PCT No.: PCT/IB2014/062644

§ 371 Date: Jan. 8, 2016

Related U.S. Application Data
(60) Provisional application No. 61/844,073, filed on Jul. 9, 2013.

(51) Int. Cl. A61M 15/00; A61M 11/00

(52) U.S. Cl. CPC A61M 15/0065; A61M 11/005; A61M 2205/52

(57) ABSTRACT

Systems and methods for monitoring and/or data-logging nebulizer usage and/or other parameters pertinent to a patient use a conduit that is fluidly coupled between a pressure source, such as a compressor, and a nebulizer, such as a jet nebulizer. During use of the nebulizer by a patient, a compressed medium such as air is transferred through the conduit, thereby altering the pressure level within the conduit. Changes in pressure level may be measured and/or detected, and subsequently used to determine one or more nebulizer usage parameters. These nebulizer usage parameters may in turn be recorded and/or analyzed.

### BACKGROUND

1. Field

The present disclosure pertains to systems and methods that monitor actual nebulizer usage and, in particular, systems and methods that store and/or analyze information derived from such monitoring.

2. Description of the Related Art

Respiratory therapy delivery devices include respiratory drug delivery devices. Respiratory therapy delivery devices are used to treat many types of patients. As used herein, respiratory drug delivery devices may be referred to as respiratory medicament delivery devices. Patient adherence is a key factor in obtaining positive treatment outcomes. For some types of respiratory drug delivery devices, for example nebulizers, information about actual usage may depend on the patient's testimony.

### SUMMARY

One or more embodiments disclosed herein provide a system configured to monitor nebulizer usage. The system comprises a conduit, a pressure transduction subsystem, and one or more processors. The conduit is configured to fluidly couple a pressure source and a nebulizer such that a compressed medium is transferred from the pressure source through the conduit to the nebulizer. The pressure transduction subsystem is configured to generate an output signal. The pressure transduction subsystem is configured to transduce pressure transferred through the conduit. The pressure transduction subsystem is configured to adjust the output signal responsive to a change in the transduced pressure. The one or more processors are configured to execute computer program modules. The computer program modules comprise a parameter determination module configured to determine one or more nebulizer usage parameters based on the output signal and a data logging module configured to store the one or more nebulizer usage parameters.

It is yet another aspect of one or more embodiments to provide a method of monitoring nebulizer usage. The method comprises transferring, through fluid coupling by a conduit, a compressed medium from a pressure source to a nebulizer; transducing, by a pressure transduction subsystem, pressure from the compressed medium into an output signal such that the output signal is adjusted responsive to a change in the transduced pressure; determining one or more nebulizer usage parameters based on the output signal; and storing the one or more nebulizer usage parameters.

It is yet another aspect of one or more embodiments to provide a system configured to monitor nebulizer usage. The system comprises means for transferring, through fluid coupling, a compressed medium from a pressure source to a nebulizer; means for transducing pressure from the compressed medium into an output signal such that the output signal is adjusted responsive to a change in the transduced pressure; means for determining one or more nebulizer usage parameters based on the output signal; and means for storing the one or more nebulizer usage parameters.

These and other aspects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of any limits.

### BRIEF DESCRIPTION OF THE DRAWINGS

FIG. 1 schematically illustrates a system configured to monitor nebulizer usage;

FIG. 2 illustrates a method of monitoring nebulizer usage;

FIGS. 3-8 illustrate systems configured to monitor nebulizer usage in accordance with various embodiments described herein; and

FIGS. 9 and 10 illustrate graphs for pressure and/or energy emitted during the operation of a nebulizer.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

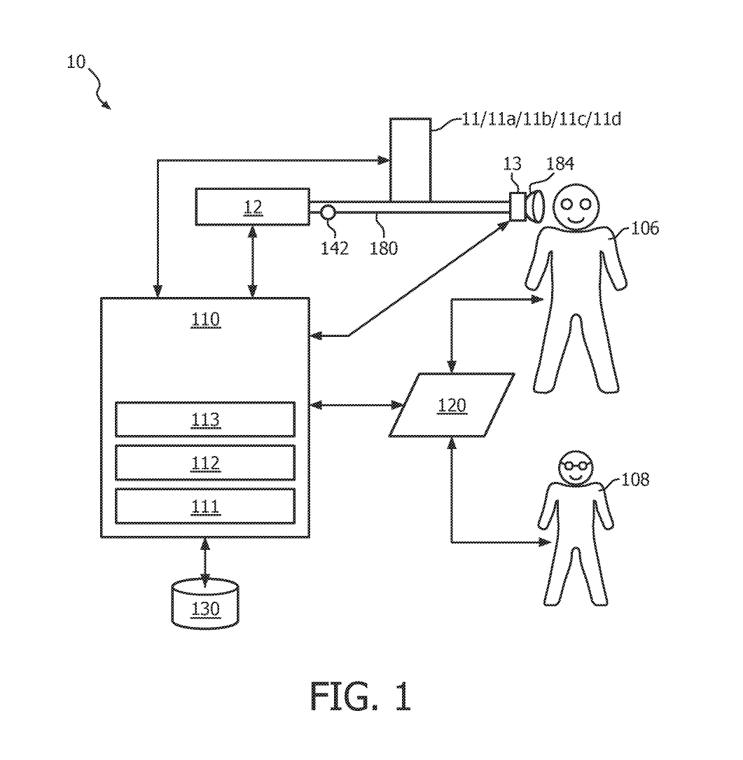

FIG. 1 schematically illustrates a system 10 configured to monitor nebulizer usage, e.g. nebulizer usage by a subject 106. System 10 may be included in, integrated in, embedded in, combined with, and/or otherwise operate conjointly with one or more devices including but not limited to devices for respiratory drug delivery, devices that provide oxygen, (positive) airway pressure devices, humidification systems, devices that aid patients with sleeping, devices that provide ventilation and/or other types of respiratory therapy devices. System 10 may include one or more of a conduit 180, a pressure source 12, a nebulizer 13, a pressure transduction subsystem 11, one or more sensors 142, one or more processors 110, a parameter determination module 111, a data logging module 112, an analysis module 113, an electronic storage 130, a user interface 120, and/or other components and/or computer program modules.

Alternatively, and/or simultaneously, system 10 may include one or more of a jet nebulizer, a mesh nebulizer, an ultrasonic wave nebulizer, a nebulizer, an aerosol generator, and/or another device configured to deliver medicament to a subject through, at least in part, respiration of subject 106. In some implementations, system 10 may include one or more features of any of these devices. For example, system 10 may be configured to combine breathable gas, e.g. air, and medicament, e.g. liquid and/or aerosolized drugs, for delivery to the airway of subject 106. In some implementations, system 10 may be operated by a care provider 108, e.g. a medical professional. By way of non-limiting example, drug delivery devices may be used to provide treatment for asthma, chronic obstructive pulmonary disease (COPD), and/or other diseases pertaining to respiratory function.

Conduit 180 may be configured to deliver, transfer, and/or guide a medium, liquid, gas and/or medicament (e.g. through fluid coupling) from pressure source 12 to nebulizer 13, subject interface appliance 184 (e.g. a mouthpiece or mask), and/or the airway of subject 106. A (compressed and/or pressurized) medium may be transferred, by and/or through conduit 180, from pressure source 12 to nebulizer 13 and/or another device configured to deliver medicament to a subject through, at least in part, respiration of subject 106.

In some implementations, pressure source 12 may include a compressor. In some implementations, e.g. in some hospital environments, pressure source 12 may be a socket, port, connector, and/or plug through which pressurized gas and/or air may be provided. Such a pressure source may be referred to as “wall air.”

Pressure transduction subsystem 11 is configured to transduce pressure (and/or a unit and/or quantity related to and/or derived from pressure) into an output signal and/or output actuation (which may be jointly referred to as output signal) such that a change in the output signal correspond to, relates to, and/or is responsive to a change in the pressure transferred through conduit 180. Different implementations of pressure transduction subsystem 11 are labeled in FIG. 1 as 11a, 11b, 11c, and 11d, and described in more detail elsewhere within this disclosure.

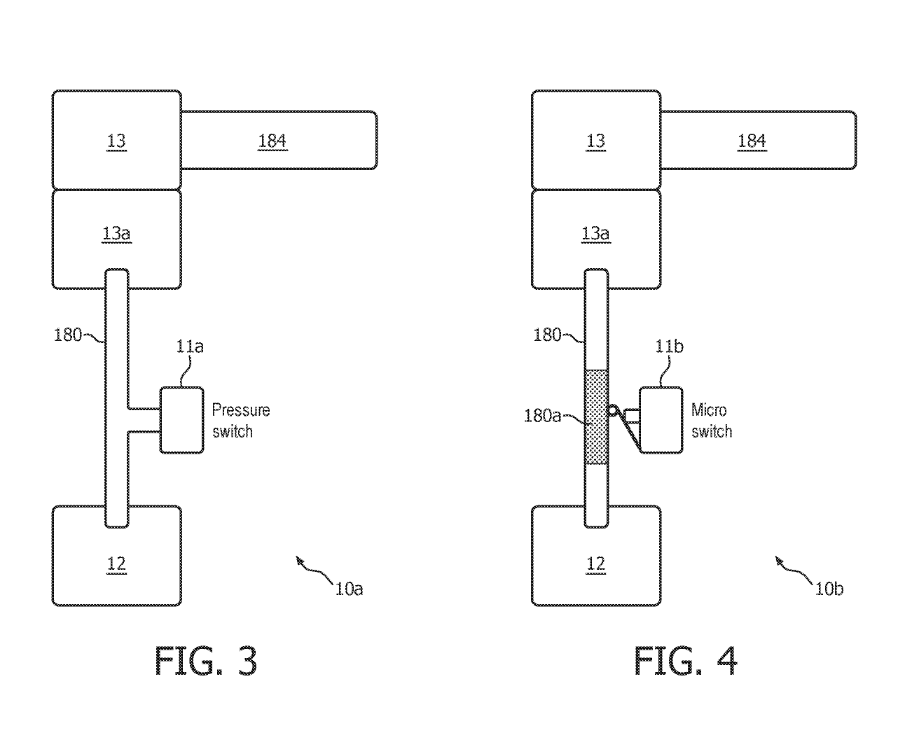

In some implementations, pressure transduction subsystem 11 may include and/or be implemented as a pressure switch 11a. Pressure switch 11a may be a mechanical switch. Pressure switch 11a may be configured to adjust an output signal (e.g. from on to off or vice versa) responsive to the level of pressure within conduit 180 breaching a pressure threshold. In some implementations, the output signal of pressure switch 11a may be used to control data logging module 112. For example, a data-logging function may be turned off responsive to insufficient pressure reaching pressure switch 11a. Alternatively, and/or simultaneously, a data-logging function may be turned on responsive to sufficient pressure reaching pressure switch 11a. By way of illustration, FIG. 3 illustrates a system 10a similar to or the same as system 10 (in FIG. 1). System 10a in FIG. 3 includes one or more of pressure source 12, conduit 180, nebulizer 13, drug fluid reservoir 13a, subject interface appliance 184 (e.g. a mouthpiece), pressure switch 11a, and/or other components. Conduit 180 may fluidly couple pressure source 12 to one or both of nebulizer 13 and/or drug fluid reservoir 13a. Pressure and/or pressurized media may be transferred from pressure source 13 through conduit 180 to one or both of nebulizer 13 and/or drug fluid reservoir 13a. Responsive to the pressure level that reaches pressure switch 11a breaching a pressure threshold, the output signal of pressure switch 11a may transition (e.g. from off to on). This output signal may be used elsewhere in system 10a, for example by other system components (not depicted). In some implementations, pressure switch 11a may be positioned and/or arrange in-line with conduit 180.

In some implementations, pressure transduction subsystem 11 may include and/or be implemented as a conduit portion 180a and a micro-switch 11b. Conduit portion 180a may be part of, integrated with, embedded in, and/or otherwise combined with conduit 180. In some implementations, conduit portion 180a may be configured to mechanically displace, move, and/or extend responsive to application of pressure, e.g. from within conduit 180 and/or conduit portion 180a. The motion of conduit portion 180a may positively correlate to the level of pressure applied. In some implementations, conduit portion 180a may include soft tubing (for example, tubing that is softer than the rest of conduit 180). The motion of conduit portion 180a may actuate micro-switch 11b (e.g. transitioning from off to on or vice versa). Micro-switch 11b may, during operation, include different states or modes of operation, including, by way of non-limiting example, an “on” state or mode, and an “off” state of mode. The state or mode of micro-switch 11b may function as the output signal of pressure transduction subsystem 11.

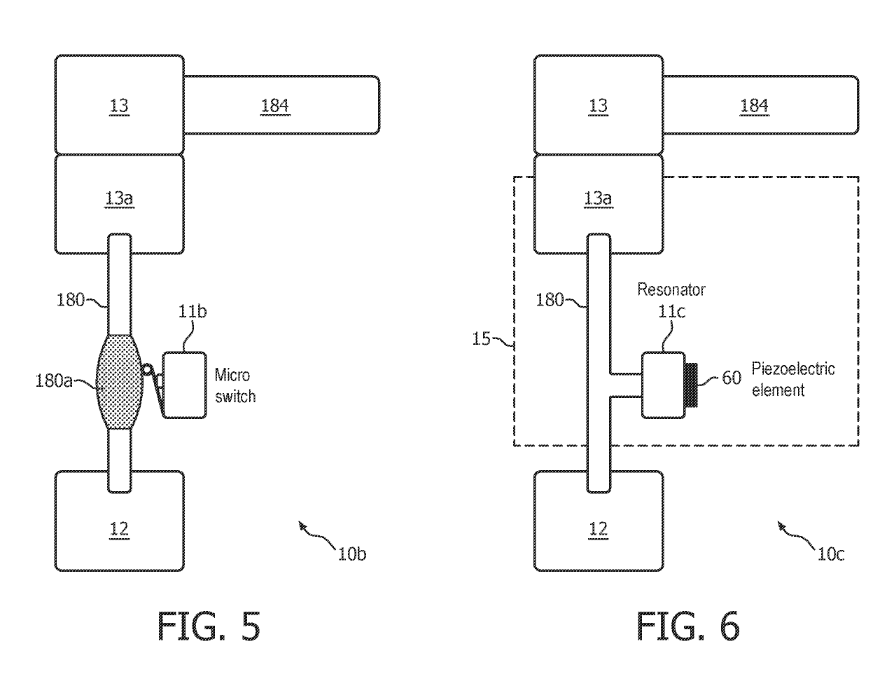

By way of illustration, FIG. 4 and FIG. 5 illustrate a system 10b similar to or the same as system 10 (in FIG. 1). System 10b in FIG. 4 and FIG. 5 includes one or more of pressure source 12, conduit 180, conduit portion 180a, nebulizer 13, drug fluid reservoir 13a, subject interface appliance 184 (e.g. a mouthpiece), micro-switch 11b, and/or other components. Conduit 180 may fluidly couple pressure source 12 to one or both of nebulizer 13 and/or drug fluid reservoir 13a. Pressure and/or pressurized media may be transferred from pressure source 13 through conduit 180 and conduit portion 180a to one or both of nebulizer 13 and/or drug fluid reservoir 13a. Responsive to the pressure level within conduit portion 180a failing to breach a pressure threshold, micro-switch 11b may be and/or remain in an “off” state or mode, e.g. micro-switch 11b may be not actuated, as depicted in FIG. 4. Responsive to the pressure level within conduit portion 180a breaching a pressure threshold, micro-switch lib may transition to an “on” state or mode, e.g. micro-switch 11b may be actuated, as depicted in FIG. 5. The output signal of micro-switch 11b may be used elsewhere in system 10b, for example by other system components (not depicted). In some implementations, micro-switch 11b may be positioned and/or arrange in-line with conduit 180.

In some implementations, pressure transduction subsystem 11 may include and/or be implemented as a resonator 11c and/or a piezoelectric element 60 (e.g. a piezoelectric disk). Resonator 11c may be an acoustic resonator. Resonator 11c may be a passive device that resonates under certain conditions, which may depend, at least in part, on the shape and/or dimensions of resonator 11c. Resonator 11c may be circular. Resonator 11c may include an opening and/or cavity such that pressurized media from conduit 180 may enter and/or contact resonator 11c. Resonator 11c may be configured to detect low frequency pump pulsations associated with pressure source 12. Pump pulsations may be caused by one or more pump valves, for example a pump exhaust valve, opening and closing during operation. By way of illustration, FIG. 6 illustrates a system 10c similar to or the same as system 10 (in FIG. 1). System 10c in FIG. 6 includes one or more of pressure source 12, conduit 180, nebulizer 13, drug fluid reservoir 13a, subject interface appliance 184 (e.g. a mouthpiece), a housing 15, resonator 11c, piezoelectric element 60, and/or other components. Conduit 180 may fluidly couple pressure source 12 to one or both of nebulizer 13 and/or drug fluid reservoir 13a. Pressure and/or pressurized media may be transferred from pressure source 12 through conduit 180 to one or both of nebulizer 13 and/or drug fluid reservoir 13a. In some implementations, resonator 11c may be configured to function as a pulsation sensor, e.g. by virtue of combination with piezoelectric element 60. Piezoelectric element 60 may be displaced responsive to movement by a surface of resonator 11c. Alternatively, and/or simultaneously, a microphone (including but not limited to an ultrasonic microphone, a micro-electro-mechanical system (MEMS) microphone, and/or another type of microphone, not depicted in FIG. 6) may be combined with resonator 11c to function as a pulsation sensor. Responsive to detection, by resonator 11c, of pump pulsations, an output signal of the pressure transduction subsystem may be generated and used elsewhere in system 10c, for example by other system components (not depicted). In some implementations, resonator 11c may be positioned and/or arrange in-line with conduit 180.

In some implementations, piezoelectric element 60 may include one or more wires (not depicted) configured to conduct pump pulsations. By virtue of these wires, pump pulsations may be detected independently from the existence and/or occurrence of operating conditions that produce resonance in the system and/or in resonator 11c. In some implementations, pump pulsations may be detected at frequencies that are too low or too high for operating conditions of a nebulizer. In some implementations, resonator 11c may be enclosed within housing 15, as depicted in FIG. 6. Housing 15 may enclose piezoelectric element 60, and all or part of conduit 180 and/or drug fluid reservoir 13a.

In some implementations, a signal generated by pressure transduction subsystem 11 may itself provide the actual power required to operate a data logger (and/or datalogging module 112).

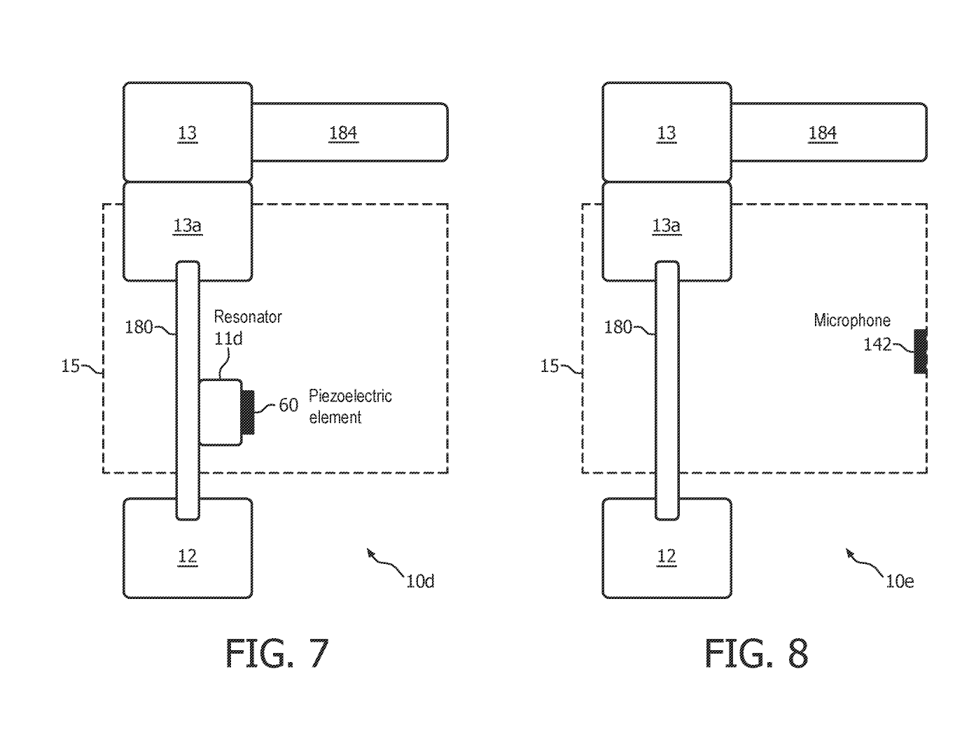

In some implementations, pressure transduction subsystem 11 may include and/or be implemented as a resonator 11d and/or a piezoelectric element 60 (e.g. a piezoelectric disk). Resonator 11d may be an acoustic resonator. Resonator 11d may be positioned and/or arranged in proximity to conduit 180 without direct contact or fluid coupling between conduit 180 and resonator 11d. For example, resonator 11d may be implemented using a cavity that is placed near conduit 180. Resonator 11d may be configured to detect low frequency pump pulsations associated with pressure source 12. By way of illustration, FIG. 7 illustrates a system 10d similar to or the same as system 10c (in FIG. 6). System 10d in FIG. 7 includes one or more of pressure source 12, conduit 180, nebulizer 13, reservoir 13a, subject interface appliance 184 (e.g. a mouthpiece), housing 15, resonator 11d, piezoelectric element 60, and/or other components. Conduit 180 may fluidly couple pressure source 12 to one or both of nebulizer 13 and/or drug fluid reservoir 13a. Pressure and/or pressurized media may be transferred from pressure source 13 through conduit 180 to one or both of nebulizer 13 and/or drug fluid reservoir 13a. In some implementations, resonator 11d may be configured to function as a pulsation sensor, e.g. by virtue of combination with piezoelectric element 60. Alternatively, and/or simultaneously, a microphone (including but not limited to an ultrasonic microphone, a micro-electro-mechanical system (MEMS) microphone, and/or another type of microphone, not depicted in FIG. 7) may be combined with resonator 11d to function as a pulsation sensor. Responsive to detection, by resonator 11d, of pump pulsations, an output signal of the pressure transduction subsystem may be generated and used elsewhere in system 10d, for example by other system components (not depicted).

In some implementations, piezoelectric element 60 may include one or more wires (not depicted) configured to conduct pump pulsations. In some implementations, resonator 11d may be enclosed within housing 15, as depicted in FIG. 7. Housing 15 may enclose piezoelectric element 60, and all or part of conduit 180 and/or drug fluid reservoir 13a.

In some implementations, pressure transduction subsystem 11 may include and/or be implemented as a microphone 142 (in particular a MEMS microphone). By way of illustration, FIG. 8 illustrates a system 10e similar to or the same as system 10 (in FIG. 1). System 10e in FIG. 8 includes one or more of pressure source 12, conduit 180, nebulizer 13, drug fluid reservoir 13a, subject interface appliance 184 (e.g. a mouthpiece), housing 15, microphone 142, and/or other components. Conduit 180 may fluidly couple pressure source 12 to one or both of nebulizer 13 and/or drug fluid reservoir 13a. Pressure and/or pressurized media may be transferred from pressure source 13 through conduit 180 to one or both of nebulizer 13 and/or drug fluid reservoir 13a. In some implementations, microphone 142 may include one or more wires (not depicted) configured to conduct acoustic pulsations. Microphone 142 may be configured to detect acoustic energy associated with nebulization and/or emitted by conduit 180. In some implementations, microphone 142 may be enclosed within housing 15, as depicted in FIG. 8. Housing 15 may enclose and/or surround all or part of conduit 180 and/or drug fluid reservoir 13a. Signals generated by microphone 142 may be used elsewhere in system 10e, for example by other system components (not depicted).

By virtue of this disclosure, actual usage of a nebulizer may be detected, measured, recorded, stored, analyzed, and/or otherwise used in a system configured to monitor nebulizer usage. Alternatively, and/or simultaneously, (patient-specific) respiratory parameters and/or adherence parameters (e.g. as indicated through device usage information and/or device actuation information) may be monitored, recorded, stored, and/or analyzed based on pressure within the system, e.g. in conduit 180. In some implementations, device usage may be monitored without interfering with either the flow of a medium in pressure source 12, nebulizer 13, and/or conduit 180, or the operation thereof.

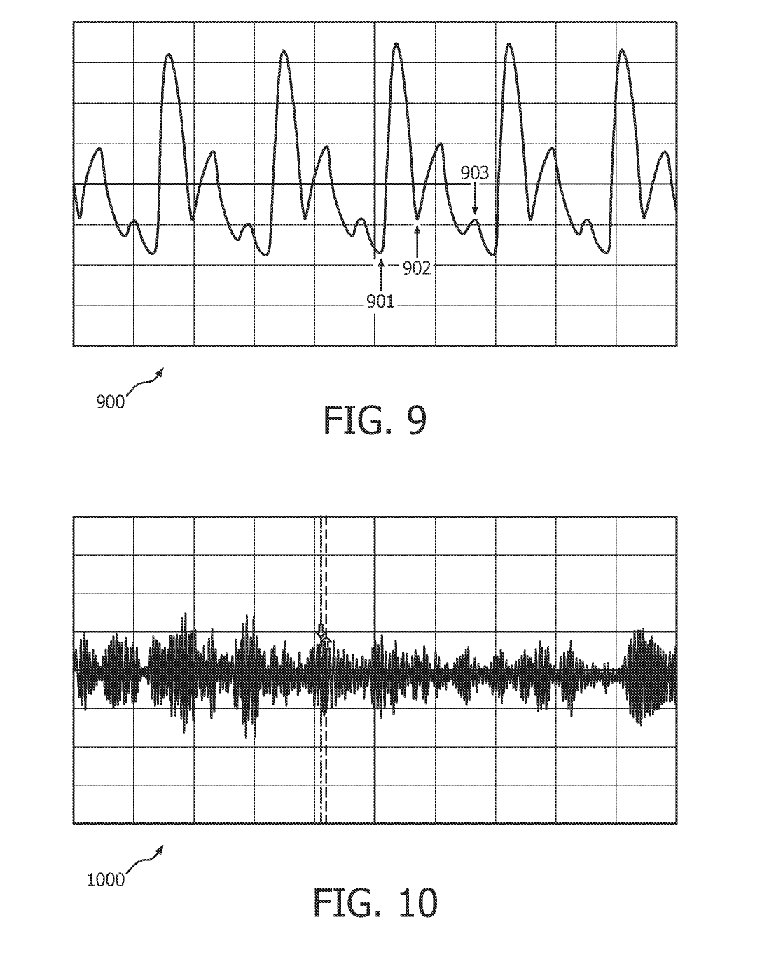

By way of illustration, FIG. 9 illustrates a graph 900 for pressure detected during the operation of a nebulizer. Graph 900 shows time on the horizontal axis and measured pressure on the vertical axis. As depicted in FIG. 9, graph 900 includes a point 901 that corresponds to the moment the discharge valve of an attached compressor opens, a point 902 that corresponds to the moment the discharge valve of an attached compressor closes, and a point 903 that corresponds to the moment the intake valve of an attached compressor closes.

By way of illustration, FIG. 10 illustrates a graph 1000 for measured ultrasonic energy during the operation of a nebulizer. Graph 1000 shows time on the horizontal axis and magnitude of measured energy on the vertical axis. As depicted in FIG. 10, graph 1000 illustrates ultrasonic energy associated with operation of nebulizer 13, of about 43 kHz.

One or more sensors 142 of system 10 in FIG. 1 are configured to generate output signals representing one or more characteristics of pressure and/or (ultrasonic) energy emitted within and/or by system 10 and/or one or more components thereof. In some implementations, sensor 142 may include a microphone (interchangeably referred to as microphone 142). For example, sensor 142 may include a microphone constructed as a micro-electro-mechanical system (MEMS) or nano-electro-mechanical system (NEMS). As used herein, the term “MEMS” may be used to refer to either MEMS or NEMS. As used in this disclosure, the term “microphone” may be used to refer to a MEMS microphone, and may be used for audible and/or ultrasonic frequencies/sounds from any source or sources that emit such energy.

The one or more sensors 142 may include an accelerometer, positional sensor, movement sensor, light sensor, infra-red (IR) sensor, electromagnetic sensor, electrode, tilt meter, (video) camera, and/or other sensors. The illustration of sensor 142 including one member in FIG. 1 is not intended to be limiting. In some embodiments, system 10 may use multiple sensors. The illustration of the location of sensor 142 as depicted in FIG. 1 is not intended to be limiting. An individual sensor 142 may be located at or near (a body part of) subject 106, embedded and/or integrated in a respiratory device, along conduit 180, and/or at other locations. Resulting output signals or conveyed information from one or more sensors 142 may be transmitted to processor 110, user interface 120, electronic storage 130, and/or other components of system 10. Transmission may be wired and/or wireless.

The one or more sensors 142 may be configured to generate output signals in an ongoing manner, e.g. before, during, and/or after delivery of medicament. This may include generating signals intermittently, periodically (e.g. at a sampling rate), continuously, continually, at varying intervals, and/or in other ways that are ongoing. The sampling rate may be about 10⁻⁹ second, about 10⁻⁸ second, about 10⁻⁷ second, 10⁻⁶ second, 10⁻⁵ second, 10⁻⁴ second, 10⁻³ second, 0.01 second, 0.1 second, 1 second, about 10 seconds, about 1 minute, and/or other sampling rates. It is noted that multiple individual sensors 142 may operate using different sampling rates, as appropriate for the particular output signals and/or (frequencies related to particular) parameters and/or characteristics derived therefrom. For example, in some embodiments, the generated output signals may be considered as a vector of output signals, such that a vector includes multiple samples of information conveyed related to one or more parameters and/or characteristics. A particular parameter or characteristic determined in an ongoing manner from a vector of output signals may be considered as a vector of that particular parameter or characteristic.

In some implementations, sensor 142 may include a MEMS microphone configured and/or arranged to measure pressure pulsations and/or energy transferred from any flat and/or curved surface within a respiratory device, any exterior surface thereof, and/or (the airway of) subject 106. For example, measured energy may be different during actual nebulization compared to, e.g., a mode of operation during which no medicament is delivered to subject 106. For example, measured energy may be different between inhalation and exhalation.

In some implementations, sensor 142 may be configured to generate output signals conveying measurements related to gas parameters of respiratory airflow, parameters related to airway mechanics, device characteristics, and/or other parameters. Gas parameters may include flow, flow rate, strength of inhalation by a patient, (airway) pressure, humidity, velocity, acceleration, and/or other gas parameters, as well as derivatives thereof. Output signals may convey measurements related to respiratory parameters, including but not limited to respiratory timing and respiratory rate.

Respiratory timing may include one or more of onset of inhalation, duration of inhalation, onset of respiratory pause between inhalation and exhalation, duration of respiratory pause, onset of exhalation, duration of exhalation, respiratory rate, inhalation-to-exhalation ratio (I:E ratio), device usage information, and/or other timing characteristics related to respiration.

Device characteristics may include characteristics of a pressure source, RPM of a compressor, characteristics of a nebulizer, mode of operation of a nebulizer, and/or other device characteristics. Sensor 142 may be in fluid communication with conduit 180 and/or mouthpiece or mask 184. Sensor 142 may generate output signals related to physiological parameters pertaining to subject 106. Parameters may be associated with the state and/or condition of an airway of subject 106, the breathing of subject 106, the gas breathed by subject 106, the composition of the gas breathed by subject 106, the delivery of the gas to the airway of subject 106, and/or a respiratory effort by the subject.

Referring to FIG. 1, electronic storage 130 of system 10 comprises electronic storage media that electronically stores information. The electronic storage media of electronic storage 130 may include one or both of system storage that is provided integrally (i.e., substantially non-removable) with system 10 and/or removable storage that is removably connectable to system 10 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Electronic storage 130 may store software algorithms, information determined by processor 110, information received via user interface 120, and/or other information that enables system 10 to function properly. For example, electronic storage 130 may record or store vectors of parameters based on the generated output signals, and/or other parameters (as discussed elsewhere herein), and/or other information. Electronic storage 130 may be a separate component within system 10, or electronic storage 130 may be provided integrally with one or more other components of system 10 (e.g., processor 110).

User interface 120 of system 10 in FIG. 1 is configured to provide an interface between system 10 and a user (e.g., a user 108, subject 106, a caregiver, a therapy decision-maker, etc.) through which the user can provide information to and receive information from system 10. This enables data, results, and/or instructions and any other communicable items, collectively referred to as “information,” to be communicated between the user and system 10. An example of information that may be conveyed by user 108 to system 10 is patient-specific adherence information. An example of information that may be conveyed to user 108 is a report detailing adherence information for subject 106. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may be provided to user 108 or subject 106 by user interface 120 in the form of auditory signals, visual signals, tactile signals, and/or other sensory signals.

It is to be understood that other communication techniques, either hard-wired or wireless, are also contemplated herein as user interface 120. For example, in one embodiment, user interface 120 may be integrated with a removable storage interface provided by electronic storage 130. In this example, information is loaded into system 10 from removable storage (e.g., a smart card, a flash drive, a removable disk, etc.) that enables the user(s) to customize system 10. Other exemplary input devices and techniques adapted for use with system 10 as user interface 120 include, but are not limited to, an RS-232 port, RF link, an IR link, modem (telephone, cable, Ethernet, internet or other). In short, any technique for communicating information with system 10 is contemplated as user interface 120.

Processor 110 of system 10 in FIG. 1 is configured to provide information processing capabilities in system 10. As such, processor 110 includes one or more of a digital processor, an analog processor, a digital circuit designed to process information, an analog circuit designed to process information, and/or other mechanisms for electronically processing information. Although processor 110 is shown in FIG. 1 as a single entity, this is for illustrative purposes only. In some embodiments, processor 110 includes a plurality of processing units.

As is shown in FIG. 1, processor 110 is configured to execute one or more computer program modules. The one or more computer program modules include one or more of parameter determination module 111, data logging module 112, analysis module 113, and/or other modules. Processor 110 may be configured to execute modules 111-113 by software; hardware; firmware; some combination of software, hardware, and/or firmware; and/or other mechanisms for configuring processing capabilities on processor 110.

It should be appreciated that although modules 111-113 are illustrated in FIG. 1 as being co-located within a single processing unit, in embodiments in which processor 110 includes multiple processing units, one or more of modules 111-113 may be located remotely from the other modules. The description of the functionality provided by the different modules 111-113 described herein is for illustrative purposes, and is not intended to be limiting, as any of modules 111-113 may provide more or less functionality than is described. For example, one or more of modules 111-113 may be eliminated, and some or all of its functionality may be incorporated, shared, integrated into, and/or otherwise provided by other ones of modules 111-113. Note that processor 110 may be configured to execute one or more additional modules that may perform some or all of the functionality attributed below to one of modules 111-113.

Parameter determination module 111 of system 10 in FIG. 1 is configured to determine one or more parameters from output signals generated by pressure transduction subsystem 11 and/or sensor(s) 142. The one or more parameters may include one or more nebulizer usage parameters, and/or other parameters. For example, nebulizer usage parameters may include nebulizer mode of operation, pressure levels within system 10, duration of treatment, estimated dosage per treatment, frequency of usage, nebulizer type, and/or other parameters.

Operation of parameter determination module 111 may be performed in an ongoing manner, for example at a particular sampling rate. The one or more parameters may be determined at different locations and/or positions within system 10 or near subject 106. In some embodiments, parameter determination module 111 may derive vectors of parameters in an ongoing manner during a period of monitoring subject 106. The vectors of the parameters may be based on vectors of generated output signals and/or other (vectors of) determined parameters.

Data logging module 112 may be configured to store one or more nebulizer usage parameters, and/or other information. Data logging module may be configured to store information in physical storage media, for example electronic storage 130. In some implementations, data logging module 112 may be implemented by a data logger, which may be a separate and independent device from the other components of system 10 described herein. Data logging module 112 may be configured to log, record, and/or store a time and/or date with any of the parameters as determined by parameter determination module 111. By combining operations of parameter determination module 111 and data logging module 112, the stored information may reflect nebulizer usage information per day, per week, per month, and/or in other manners that may be pertinent to derive long-term (trend) information about a patient's treatment and/or changes in medical conditions. For example, the stored information may reflect whether a patient is under-dosing, overdosing, delay-dosing, and/or otherwise not following his treatment plan properly.

In some implementations, operation of and/or power supply to data logging module 112 may be based on one or more output signals generated by pressure transduction subsystem 11, as described elsewhere herein. For example, if pressure measurements indicate nebulizer 13 is not being used, then data logging module 112 may be turned off, powered down, and/or otherwise transitioned into a non-operational mode.

Analysis module 113 may be configured to determine whether subject 106 has been following his treatment plan. Analysis module 113 may be configured to retrieve information stored by data logging module 112, and perform operations, processing, and/or analysis thereupon. For example, analysis by analysis module 113 may reveal that subject 106 has been increasing the dosage and/or treatment time, which may be related to an exacerbation of certain symptoms, a particular disease, and/or illness of subject 106.

In some implementations, analysis module 113 may be configured to determine an adherence metric and/or an adherence parameter for subject 106. The adherence metric and/or adherence parameter may be based on one or more previously described parameters and/or characterizations. For example, a particular adherence metric may be based on a combination of, at least, device actuation information and respiratory timing. An adherence metric and/or adherence parameter may for example be expressed as a percentage of perfect compliance with the recommended treatment. For example, if a particular patient scored a 90% adherence, such a score that may be considered by a care giver in determining a course of action. Alternatively, if a particular patient scored a low percentage of adherence, such a score may be considered relevant before the particular drug is deemed ineffective for that particular patient. Low scores may, for example, prompt a change in the chosen type of respiratory device.

In some implementations, one or more of the various types of pressure transduction subsystems 11/11a/11b/11c/11d and/or microphone 142 (and combinations thereof) may be used in pneumatic medical devices that include application of pressure to one or more accessible tubes.

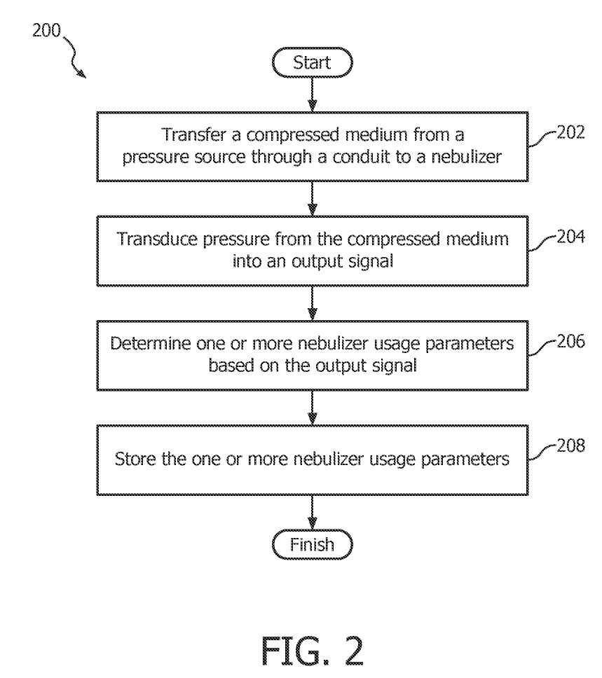

FIG. 2 illustrates a method 200 to monitor nebulizer usage. The operations of method 200 presented below are intended to be illustrative. In certain embodiments, method 200 may be accomplished with one or more additional operations not described, and/or without one or more of the operations discussed. Additionally, the order in which the operations of method 200 are illustrated in FIG. 2 and described below is not intended to be limiting.

In certain embodiments, method 200 may be implemented in one or more processing devices (e.g., a digital processor, an analog processor, a digital circuit designed to process information, an analog circuit designed to process information, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, a compressed medium is transferred, through fluid coupling by a conduit, from a pressure source to a nebulizer. In some embodiments, operation 202 is performed by a conduit the same as or similar to conduit 180 (shown in FIG. 1 and described herein).

At an operation 204, pressure from the compressed medium is transduced into an output signal such that the output signal is adjusted responsive to a change in the transduced pressure. In some embodiments, operation 204 is performed by a pressure transduction subsystem the same as or similar to pressure transduction subsystem 11 (shown in FIG. 1 and described herein).

At an operation 206, one or more nebulizer usage parameters are determined based on the output signal. In some embodiments, operation 206 is performed by a parameter determination module the same as or similar to parameter determination module 111 (shown in FIG. 1 and described herein).

At an operation 208, the one or more nebulizer usage parameters are stored, e.g. in physical storage media. In some embodiments, operation 208 is performed by a data logging module the same as or similar to data logging module 112 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although this description includes details for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that, to the extent possible, one or more features of any embodiment are contemplated to be combined with one or more features of any other embodiment.

1. A system configured to monitor nebulizer usage, the system comprising:
a conduit configured to fluidly couple a pressure source and a nebulizer such that a compressed medium is transferred from the pressure source through the conduit to the nebulizer, wherein the conduit includes a portion configured to mechanically displace responsive to application of pressure, wherein such displacement does not substantially interfere with transfer through the conduit;
a pressure transduction sub system configured to generate an output signal, wherein the pressure transduction subsystem is configured to transduce pressure transferred through the conduit, wherein the pressure transduction subsystem includes a micro-switch positioned along the portion included in the conduit such that displacement of the portion actuates the micro-switch, wherein the pressure transduction subsystem is further configured to adjust the output signal responsive to actuation of the micro-switch; and
one or more processors configured to execute computer program modules, the computer program modules comprising:
a parameter determination module configured to determine one or more nebulizer usage parameters based on the output signal; and
a data logging module configured to store the one or more nebulizer usage parameters.

2. The system of claim 1, wherein the pressure transduction subsystem includes a pressure switch configured to control the data logging module through the output signal.

3. The system of claim 1, wherein the micro-switch is configured to include different states responsive to actuation, wherein the different states include an “on”-state and an “off”-state.

4. The system of claim 1, wherein the computer program modules further comprise an analysis module configured to retrieve the one or more nebulizer usage parameters, and wherein the analysis module is further configured to determine whether usage of the nebulizer is in accordance with a treatment plan, wherein the determination is based on the retrieved one or more nebulizer usage parameters.

5. The system of claim 4, wherein the analysis module is further configured to determine whether usage of the nebulizer has increased based on the retrieved one or more nebulizer usage parameters.

6. A method of activating the monitoring of nebulizer usage, the method comprising:
transferring, through fluid coupling by a conduit, a compressed medium from a pressure source to a nebulizer, wherein the conduit includes a portion that mechanically displaces responsive to application of pressure, wherein such displacement does not substantially interfere with transfer through the conduit;
positioning a micro-switch along the portion of the conduit such that displacement of the portion actuates the micro-switch,
transducing, by a pressure transduction subsystem, pressure from the compressed medium via the portion of the conduit into an output signal such that the output signal is adjusted responsive to actuation of the micro-switch;
determining one or more nebulizer usage parameters based on the output signal; and
storing the one or more nebulizer usage parameters.

7. The method of claim 6, wherein the pressure transduction subsystem includes a pressure switch, and wherein storing the one or more nebulizer usage parameters is controlled based on the output value of the pressure switch.

8. The method of claim 6, wherein the micro-switch includes different states responsive to actuation, wherein the different states include an “on”-state and an “off”-state.

9. The method of claim 6, further comprising:
retrieving the one or more nebulizer usage parameters; and
determining whether usage of the nebulizer is in accordance with a treatment plan, wherein the determination is based on the retrieved one or more nebulizer usage parameters.

10. The method of claim 6, further comprising:
retrieving the one or more nebulizer usage parameters, and
determining whether usage of the nebulizer has increased based on the retrieved one or more nebulizer usage parameters.

11. A system configured to monitor nebulizer usage, the system comprising;
means for transferring, through fluid coupling, a compressed medium from a pressure source to a nebulizer, wherein the means for transferring includes a portion that mechanically displaces responsive to application of pressure, wherein such displacement does not substantially interfere with transfer through the means for transferring;
micro-switch means for positioning along the portion such that displacement of the portion actuates the micro-switch means;
means for transducing pressure from the compressed medium via the portion into an output signal such that the output signal is adjusted responsive to actuation of the micro-switch means;
means for determining one or more nebulizer usage parameters based on the output signal; and
means for storing the one or more nebulizer usage parameters.

12. The system of claim 11, wherein the means for transducing includes a pressure switch, and wherein operation of the means for storing is controlled based on the output value of the pressure switch.

13. The system of claim 11, wherein the micro-switch means includes different states responsive to actuation, wherein the different stales include an “on”-state and an “off”-state.

14. The system of claim 11, further comprising: means for retrieving the one or more nebulizer usage parameters and determining whether usage of the nebulizer is in accordance with a treatment plan, wherein the determination is based on the retrieved one or more nebulizer usage parameters.

15. The system of claim 11, further comprising: means for retrieving the one or more nebulizer usage parameters and determining whether usage of the nebulizer has increased based on the retrieved one or more nebulizer usage parameters.
